(19) United States
(12) Patent Application Publication
(10) Pub. No.: US 2011/0098564 A1
(43) Pub. Date: Apr. 28, 2011

(54) Method and apparatus for diagnosing and treating vascular disease

(76) Inventors: Marian L. Larson, Bellingham, WA (US); Eugene A. Larson, Lummi Island, WA (US)

(21) Appl. No.: 12/925,611

(22) Filed: Oct. 26, 2010

Related U.S. Application Data
(60) Provisional application No. 61/279,832, filed on Oct. 26, 2009.

(51) Int. Cl. A61B 8/14

(52) U.S. Cl. 600/439; 600/440

(57) ABSTRACT

Imaging and therapy for the treatment of venous disease are integrated into the same device, such that there is a common user interface and/or display, which may be passive or interactive between image and therapy parameters.

This application claims the benefit of U.S. Provisional Patent Application No. 61/279,832 filed Oct. 26, 2009, the entire disclosure of which is hereby incorporated by reference.

### FIELD OF THE INVENTION

The present invention is directed to the treatment of vascular disease and in particular discloses a method and apparatus wherein imaging and therapy are integrated into a single device.

### BACKGROUND OF THE INVENTION

The application of ultrasound imaging is expanding rapidly in today's society as a diagnostic test used to evaluate the health of blood vessels, in particular peripheral vascular disease. Imaging, evaluation, treatment and subsequent monitoring of diseases such as varicose veins and deep vein thrombosis (DVT) can often be performed as an outpatient procedure. What once was performed only in hospitals is now being utilized in physician offices, vascular suites, and even vein clinics. Vascular ultrasound is used for both diagnoses of vascular disease and to evaluate therapeutic potential and outcome.

To augment the diagnosis and evaluation of vascular disease and to help plan treatment, duplex ultrasound is often used, which combines Doppler flow information and conventional 2D imaging information, sometimes called B-mode, to allow physicians to see the anatomical structure of blood vessels, image and diagnose vascular disease, and to image and quantify blood flow.

Duplex vascular ultrasound imaging is useful for investigating many symptoms of vascular disease such as:

1. To investigate the cause of the following symptoms in an arm or leg:
Pain
Swelling
Increased warmth, or coolness of the extremity
Difficult to find pulses
Bulging veins

2. To diagnose the following:
Poor circulation due to blocked or narrowed blood vessels
Blood clots
Poor blood vessel function

Therapeutic treatment of vascular disease, for example treatment of varicose veins, traditionally has utilized a surgical approach, such as ligation and division of the saphenous trunk and all proximal tributaries, then stripping the vein.

Endovenous thermal ablation, using radiofrequency or laser energy, is often used as a replacement for surgery in the treatment of venous disease such as varicose veins. The veins are typically ablated or sealed closed.

Endovenous ablation, such as is used to treat varicose veins, is often an outpatient procedure. A local anesthetic is applied and a thin catheter is inserted into the vein and guided up the great saphenous vein in the thigh to the tributaries that are to be treated. Then energy, (including laser or radio frequency) is applied to the inside of these small veins, which heats and seals the veins closed. Once the diseased vein is closed, other healthy veins then re-establish normal blood flow to the area.

Imaging, such as duplex ultrasound, is used to guide the insertion of the catheter into the vein, to mark on the surface of the body the position of the vein, to guide the injection of anesthetic agent along the entire course of the vein, and to examine the vein after the procedure has been completed. Duplex ultrasound is also used to position the tip of the ablation catheter, for example below the level of the terminal valve of the saphenofemoral junction prior to starting an ablation procedure, in that if the catheter extends into the femoral vein it may cause injury to the femoral vein.

A therapeutic procedure, for example using high-frequency alternating current (Radio Frequency or RF energy), begins by then using a separate instrument not associated or linked to the ultrasound imaging system, to apply RF energy to the vein walls and deliver energy directly into tissue. Other technology heats the tip of the catheter, rather than the tissue, and delivers infrared energy directly to the vein walls to develop a thermal dose sufficient to seal them closed.

A typical procedure for treatment involves slowly withdrawing the therapeutic catheter from the vein, using parameters for time, temperature, impedance, current, pulse cycles, etc. established from experience. Often the catheter will be withdrawn at a specific rate, such as 1 centimeter per minute, in order to achieve and maintain the proper thermal dose.

Peripheral vascular disease, such as varicose veins, may require treatment, as mentioned above, to ablate or close some portion of the vein. In other therapeutic applications, such as deep vein thromboses, portions of the vein may require opening such as to increase the orifice of a narrowed vessel or to remove thromboses.

Deep Vein Thrombosis (DVT) occurs in veins that are located deep within surrounding muscle, such as the thigh. Blood thinners are often utilized to reduce the risk of clots forming pulmonary emboli, but do not typically remove an existing clot. Thrombolysis is a common procedure that may combine both chemical and mechanical means to remove a clot. Interventional techniques that combine drugs with macerating devices or HIFU have potential for the treatment of common and potentially dangerous blood-clotting condition, deep vein thrombosis (DVT).

Catheter-directed thrombolysis dissolves blood clots in the veins without surgery. Using ultrasound imaging, the clot is located and a thrombolytic agent is injected directly into the site of the clot (the deep vein thrombus, DVT). In this procedure, a catheter is inserted into a vein in the leg and moved to the site of the clot. When the catheter tip is in the clot, the thrombolytic agent is infused.

Traditionally, it takes a few days to completely dissolve the clot. The interventional radiologist can monitor the treatment using X-rays which can be supplemented with ultrasound imaging. After effective thrombolysis, post therapy imaging modalities facilitate evaluation of the vein wall and its structure (if the vein is narrowed or damaged, there is an increased risk of future thrombosis).

In the past few years, interventional radiologists have tried to improve catheter-directed thrombolysis or CDT to treat DVT with reduced morbidity and therapy time. New techniques have been developed which utilize the direct delivery of thrombolytic agents with devices that masticate and remove the clot or dissolve the clot simultaneously.

Many mechanical devices are on the market to help remove thromboses. One technique is the “Power-Pulse Spray”, which injects a thrombolytic agent to the clot at a high force. This procedure reduces the time to dissolve the clot. The clot is partially dissolved in a half-hour.

The vein can then be sprayed again with saline jets which break up the clot and also create a vacuum to remove the clot into the catheter. The Xpeedior RT device aspirates the debris by utilizing high velocity jets to create a localized low pressure zone.

A second technique uses the “Trellis-8 Peripheral Infusion System”, which uses an inflated balloon placed on both sides of the clot to contain the clot. A wire filament is inserted through the clot. The wire turns, breaking the clot into pieces which are then aspirated into the catheter.

A third technique is the “Helix Clot Buster Thrombectomy Device” which houses a small rotating device in the distal end. The impeller breaks the clot into small pieces that can be flushed from the body.

Other energy sources, such as SonoLysis (Ultrasound combined with micro bubbles), HIFU and laser are in development for independent and pharmacologically augmented lyses of clots.

These nonsurgical techniques, which may include mechanical, thermal, or clot-dissolving drug application, are performed using imaging (including ultrasound imaging with Doppler flow) to help place the device and assess the progress of the therapy.

Currently, there is no device that enables a physician to simultaneously image, evaluate and diagnose vascular disease and then perform treatment and outcome assessment with the same instrument, nor is there an instrument that integrates the user interface of the imaging and therapeutic modalities, either for usefulness in the operation of the system or the interactive integration to improve on optimizing a therapeutic procedure.

A combined or integrated imaging and therapy system would provide the data and feedback to significantly reduce the complications and risks associated with vascular disease treatment such as hematoma, surgical puncture, distal embolization, severe complications from sclerotherapy, including ulceration or pulmonary emboli. The greatest current area of concern is deep vein thrombosis, with one 2004 study documenting deep vein thrombus requiring anticoagulation in 16% of 73 limbs treated with a radiofrequency ablation procedure. Correct catheter placement at the initiation of the procedure is critical to reduce the risk of DVT and can be facilitated by a system that would combine the therapy with vein imaging to visualize the catheter position.

### SUMMARY OF THE INVENTION

The present invention integrates, into the same device, imaging and therapy for the treatment of venous disease, such that there is a common user interface, which may be passive or interactive between image and therapy parameters. The inventive device comprises several levels of integration between imaging and therapy, which may include:
A common user interface with integrated imaging screen where the image is generated by a stand-alone imaging device and electronically transported to the combined device controlling the therapy.
An imaging modality could be integrated into the combined imaging and therapy device with control of the image as part of the same user interface as the therapeutic device, such as a catheter.
A display unit in the inventive combined device could have imaging and therapy information interleaved or displayed intermittently.

### BRIEF DESCRIPTION OF THE DRAWINGS

FIG. 1 shows a console combining imaging and therapy modalities.

FIG. 2 depicts a portion of human anatomy, in particular a right leg showing the saphenous vein. Adjacent to the proximal side of the leg near the saphenous vein is an ultrasound imaging transducer.

FIG. 3 illustrates an image from the display of the inventive apparatus depicting an ultrasound image of the saphenous vein with a therapeutic device placed intravenously approaching a blood clot.

FIG. 4 shows a motor attached in the region of the ankle to control the advancement and withdrawal of a catheter within a vein. The motor can be controlled through the integrated system user interface in the console.

### DETAILED DESCRIPTION OF THE PREFERRED EMBODIMENTS

The present invention will be discussed with reference to the accompanying Figures which represent the invention by way of example only.

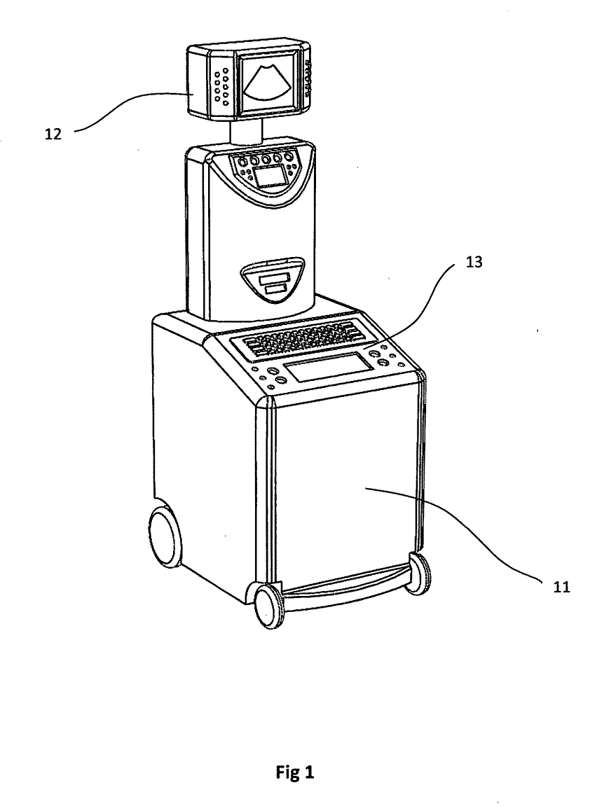

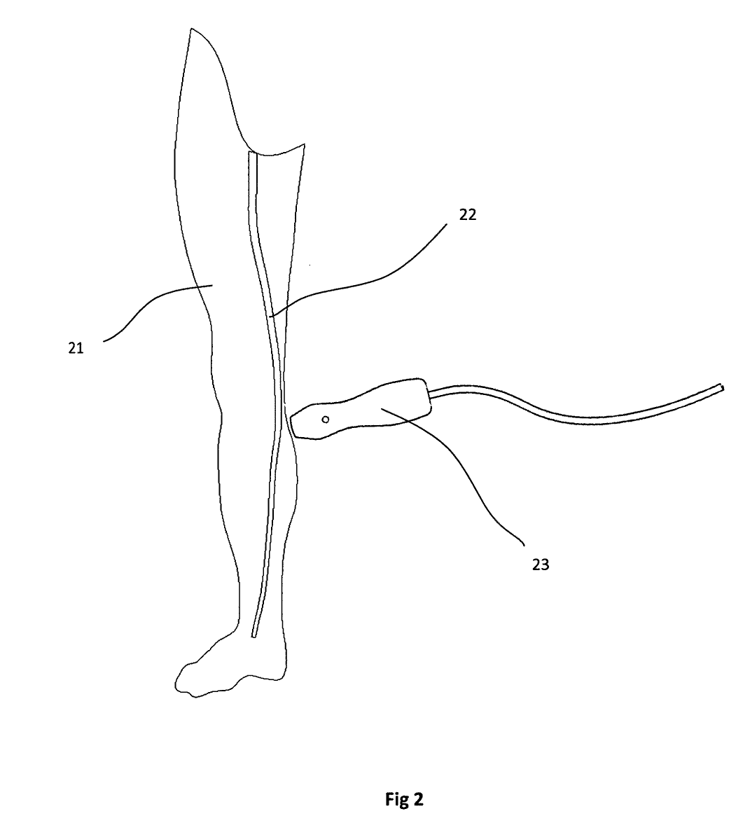

FIGS. 1 and 2 show an ultrasound imaging system with a patient contact ultrasound transducer 23 (such as a linear array) adjacent a leg for imaging a portion of a vein, such as the saphenous vein 22 of a right leg 21 and providing Doppler flow and vein diameter information to an integrated system controller located in a console unit 11. The console unit 11 includes a display 12, which can show both imaging and therapy data, and a common user interface 13. The positional and morphological information may be combined with thermal dose data and fed to a therapeutic vein ablation device, e.g. for treatment of varicose veins, or the thrombolytic device for treating blood clots.

Information, such as the position and thermal dose of a catheter, may be sent to the common display 12 and user interface 13 of the system. This data is then utilized to enhance the performance and efficacy of the therapy procedure through methods such as:
Positional and thermal dose information to the display to allow real-time adjustment and optimization of the therapy parameters, including catheter withdrawal speed.
The information could be utilized to drive a motor attached to the catheter.
A complete feedback loop could be developed whereby information from the ablation site can be fed back to the motor to automatically move the catheter and maintain a proper thermal dose.

Once the catheter is introduced into the patient, e.g. to treat varicose veins, and the ablation procedure is about to begin, the catheter can be clamped into a motor-controlled device that controls the speed at which the catheter would be moved through the vein based on Doppler flow and vein diameter information delivered from the ultrasound imaging system. This feature of the invention controls the delivery of a reproducible and consistent thermal dose to the vein.

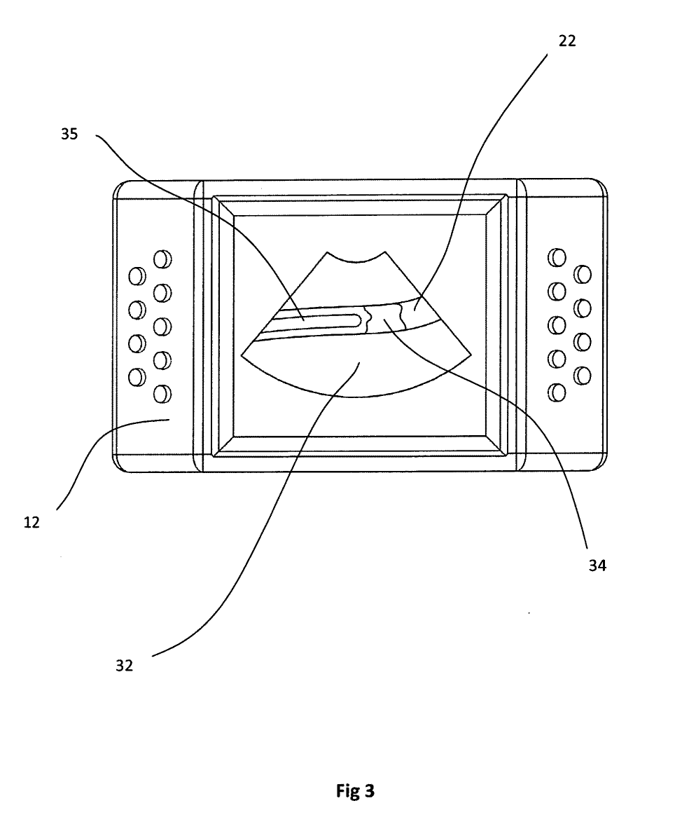

In addition to controlling the speed at which the catheter is withdrawn, the invention utilizes information provided by an imaging system to control the power that the therapeutic device is delivering to the tissue based on blood flow and temperature and the changing morphology of the venous tissue. This is illustrated in FIG. 3 which illustrates an ultrasound image 32 from the display 12 of the saphenous vein 22 with a therapeutic device 35, such as a catheter, placed intravenously approaching a blood clot 34.

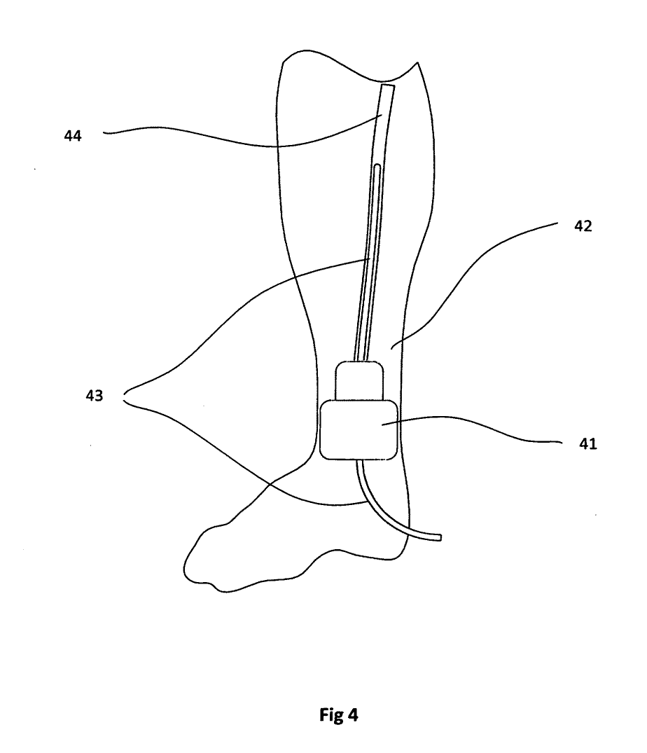

A catheter extraction motor assembly may be strapped to the patients ankle or attached to a boot-like device to insure that the limb is mechanically coupled to the vein and catheter as seen in FIG. 4. The motor assembly may include a motor 41 attached in the region of the ankle 42 to control the advancement and withdrawal of the catheter 43 within a vein 44. The motor is preferably controlled through the integrated system user interface 13 in the console 11.

Several forms of energy have been utilized for vein ablation which can be controlled by the integrated venous ultrasound imaging and ablation system, including:
Radiofrequency (Electrical Current Control)
High Intensity Focused Ultrasound (HIFU)—Ultrasound intensity control
Light (Laser)

Regardless of the energy source or catheter type, the therapeutic effect of the heat delivered to the vein causes the vessel to shrink in the treated area. Thermal denaturation and shrinkage of collagen in the vessel wall is an irreversible process beyond about 55° C. This procedure is typically performed through the entire length of the vein being treated in a “blind” manner, meaning there is no imaging of the change or shrinkage in the vein during therapy and the medical personnel must rely on experience and historical responses to provide the basis for adjusting the parameters of the therapeutic procedure.

By interfacing diagnostic imaging with the therapeutic procedure, the system can provide feedback that controls the temperature and time the catheter dwells in any one portion of the vein and assists in avoiding thermal injury to the surrounding tissues or carbonization of the vein wall.

Alternatively, High Intensity Focused Ultrasound (HIFU) can be used as a heat source to develop the proper thermal dose, either transcutaneously or by catheter delivery, to generate heat sufficient to seal the smaller varicose branched veins, as discussed above.

The passive, common user interface 13 and display 12 for image and therapy parameters greatly facilitates control of the therapeutic process as the therapy can be virtual or real time guided by the imaging and flow information incorporated into the same display or panel as the therapeutic control.

For treatment of DVT, the inventive combined imaging and therapy apparatus can be utilized to image the site of the thrombosis while simultaneously advancing and applying a thrombolytic device. The performance and efficacy of the therapeutic approach can be imaged real-time and temporal, positional, power and thrombolytic drug application can be adjusted accordingly. An example of treating DVT in the saphenous vein is shown in FIGS. 1 and 2.

With the addition of an interactive interface between the imaging (such as duplex Doppler) and therapy (such as RF Ablation), parameters such as temperature, flow, clot volume, vascular morphology and contour, dimensions etc. can be used to control the intensity and/or time of the therapeutic energy application. Knowledge based computing can be integrated into the interactive interface to provide independent quality and safety verification of pharmacological agents and energy source compatibility. Parameters of therapy based on the diameter of vessels, length of clot, type of pharmaceutical augmentation etc. can be integrated into the passive system (as management suggestions to the medical personnel) or to directly establish therapeutic parameters in an interactive user interface.

The present invention couples or integrates the imaging procedure and the therapy procedure into one device, with a common user interface, and can provide real time image and/or Doppler based information on the condition of the vein to medical personnel operating the system and/or to the therapy electronics such that the parameters of treatment can be modified or selected to achieve the therapeutic goal.

The ultrasound imaging may be performed transcutaneously, as the catheter is withdrawn, or an imaging transducer can be incorporated into the therapy catheter.

The inventive single system for imaging and treating venous disease can achieve interactive image guided therapy by the use of a microprocessor which integrates the changing morphology and contour of the vein with the therapeutic energy and the speed of the catheter withdrawal. If manual control of the therapeutic process is preferred, the invention can display real time morphology and contour changes of the vein and permit the operator to adjust the parameters of treatment, such as temperature and rate of catheter withdrawal, based on the actual real time effect on the vein.

The present invention can couple imaging to therapy, as in the example of the treatment of varicose veins above, within a single system to provide real time operator guidance or interactive therapeutic device control based on the changes in morphology and venous disease state.

As in the example above, the coupling of imaging to therapy is accomplished by providing a single user interface, a common display of image and therapeutic parameters, and a microprocessor controlled interface between the image based guidance and therapy energy source if desired.

While the invention has been described with reference to preferred embodiments it is to be understood that the invention is not limited to the particulars thereof. The present invention is intended to include modifications which would be apparent to those skilled in the art to which the subject matter pertains without deviating from the spirit and scope of the appended claims.

What is claimed is:

1. An imaging and therapeutic medical device for the treatment of vascular disease, said device comprising:
an imaging and/or Doppler flow modality to visualize the site of treatment,
a therapeutic modality comprising a therapy catheter,
wherein said imaging modality and said therapeutic modality are combined into a single device with said device having a common user interface and display.

2. The device of claim 1 wherein the imaging modality is medical ultrasound.

3. The device of claim 2 further including duplex Doppler and color flow imaging.

4. The device of claim 1 wherein the common user interface includes an integrated imaging screen with an image being generated by a stand-alone imaging device and electronically transported to the combined device controlling the therapy.

5. The device of claim 1 further comprising the imaging modality being integrated into the combined imaging and therapy device with control of the image being part of the same user interface as the therapeutic modality.

6. The device of claim 1 further comprising a display unit having imaging and therapy information interleaved or displayed intermittently.

7. The device of claim 1 wherein the ultrasound imaging comprises transcutaneous ultrasound imaging.

8. The device of claim 1 wherein the ultrasound imaging transducer is intraluminal and incorporated into the therapy catheter.

9. The device of claim 1 further including a motorized device attachable to a patient and being controllable to selectively control movement and position of the catheter in the patient.

10. The device of claim 1 wherein the catheter further provides positional and thermal dose information to the interface and display whereby real time adjustment and optimization of the therapy parameters including catheter withdrawal speed may be effected.

11. The device of claim 10 wherein said information controls driving of a motor attached to the catheter.

12. The device of claim 11 further including a feedback loop whereby information from a treatment site is fed back to the motor to automatically move the catheter and maintain a proper thermal dose.

13. A method of imaging and therapeutically treating vascular disease of a patient, said method comprising:
imaging at least a portion of a diseased vascular region to visualize a region of treatment,
inserting a therapeutic catheter into the vascular region of treatment for treating at least a portion of said region of treatment,
controlling said imaging and said therapeutic catheter via a single device with said device having a common user interface and display.

14. The method of claim 13 wherein the imaging modality is medical ultrasound.

15. The device of claim 14 further including duplex Doppler and color flow imaging.

16. The method of claim 13 further including said catheter providing positional and thermal dose information to the interface and display thereby effecting real time adjustment and optimization of the therapy parameters including catheter withdrawal speed.

17. The method of claim 16 wherein said information controls driving of a motor attached to the catheter.

18. The method of claim 17 further including feeding information from a treatment region back to the motor via a feedback loop to automatically move the catheter and maintain a proper thermal dose.

19. The method of 13 further comprising providing a motorized device being controllable to selectively control movement and position of the catheter in the patient.

20. The method of claim 14 wherein an ultrasound imaging transducer is incorporated into the therapy catheter.
